(19) United States
(12) Patent Application Publication
(10) Pub. No.: US 2015/0173975 A1
(43) Pub. Date: Jun. 25, 2015

(54) NONWOVEN, SHEET FOR ABSORBENT ARTICLE, AND ABSORBENT ARTICLE USING THE SAME

(71) Applicants: DAIWABO HOLDINGS CO., LTD., Osaka (JP); DAIWABO POLYTEC CO., LTD., Osaka (JP); THE PROCTER & GAMBLE CO., Cincinnati, OH (US)

(72) Inventors: Kosuke Harumoto, Hyogo (JP); Hiroko Makihara, Hyogo (JP); Pietro Cecchetto, Cincinnati, OH (US); Digvijay Rawat, Cincinnati, OH (US); Jan Fuhrmann-Evers, Schwalbach am Taunus (DE)

(21) Appl. No.: 14/414,770

(22) PCT Filed: Jan. 8, 2013
(86) PCT No.: PCT/CN2013/070214

§ 371 Date: Jan. 14, 2015

(30) Foreign Application Priority Data
Aug. 8, 2012 (CN) PCT/CN2012/079826

(51) Int. Cl. A61F 13/511; D04H 3/147; B32B 5/26; D04H 3/007; A61F 13/15; B32B 5/02

(52) U.S. Cl. CPC A61F 13/5116; A61F 13/15707; B32B 5/022; B32B 5/26; D04H 3/007; D04H 3/147; A61F 13/15699; A61F 2013/51178; D10B 2321/021; D10B 2509/026; B32B 2250/02; B32B 2250/20; B32B 2250/242; B32B 2262/0253; B32B 2262/12; B32B 2555/02

(57) ABSTRACT

The present invention relates to a nonwoven comprising: a first fiber layer comprising a first core/sheath composite fiber having three-dimensional crimp, which is composed of a sheath component comprising linear low density polyethylene, and a core component comprising thermoplastic resin having a high melting point, and the center of gravity of the core component being offset from the center of gravity of the fiber; a second fiber layer comprising a second core/sheath composite fiber having three-dimensional crimp, which is composed of a sheath component comprising high density polyethylene, a core component comprising thermoplastic resin having a high melting point, the center of gravity of the core component being offset from the center of gravity of the fiber; wherein at least a portion of the first and the second core/sheath composite fibers is thermal bonded via the sheath components of the first and the second core/sheath composite fibers

### FIELD OF THE INVENTION

The present invention relates to nonwoven, a method for manufacturing the same, a nonwoven sheet for an absorbent article, and also an absorbent article using the sheet.

### BACKGROUND OF THE INVENTION

Nonwovens including synthetic fibers formed from thermoplastic resin are widely used as sheets of absorbent articles such as sanitary napkins, infant disposable diapers, personal care disposable diapers, and the like. Various nonwovens have been suggested for use as sheets such as topsheets for absorbent articles from the standpoints of skin sensation, a feeling of dryness, comfort, absorption of expelled bodily fluids, and prevention of fluid flow-back.

Japanese Unexamined Patent Publication No. 2001-315239 discloses a laminate nonwoven fabric for bags, container lids and water-proof moisture-permeable clothes comprising a heat sealing layer and a non-heat sealing layer wherein the heat sealing and non-heat sealing layers include core/sheath composite fibers, and the layers are integrated by fusing sheath component via thermal treatment using a heat roller. Japanese Patent No. 3048400 discloses a nonwoven fabric prepared by piling (A) a nonwoven web of a long synthetic conjugate fiber of core/sheath type composed of core component of a polymer having higher melting point than that of sheath component polymer, and sheath component of a polymer, and (B) a nonwoven web of a long synthetic conjugate fiber of core/sheath type composed of core component of a polymer having higher melting point than that of sheath component polymer, and sheath component of a polymer having higher melting point than that of sheath component polymer of the long synthetic conjugate fiber included in the web (A); to give a laminate and pressing it with heating. Obtained nonwoven fabric with both sides surfaces having long fibers has improved abrasion resistance and is suggested for making e.g. bag by pressing with heating. Japanese Unexamined Patent Publication No. 2006-233364 describes a nonwoven comprising a first layer having a first surface and a second layer having a second surface, wherein the density of the second layer is less than the density of the first layer, and the nonwoven is produced using an air-through process. In this nonwoven, at least the fiber included in the first layer have a cross-section that is flat, and a major axis of said cross-section is oriented in a direction that is substantially parallel to a surface of the nonwoven.

There is a need for a nonwoven with improved surface smoothness. There is also a need for an absorbent article that provides improved tactile sensation and a feeling of dryness and comfort. In particular, it has not been possible to obtain a nonwoven topsheet for an absorbent article having feathery softness when contacting the skin, a luxurious tactile sensation, an appropriate amount of cushioning and a desirable bulkiness.

### SUMMARY OF THE INVENTION

The present invention provides a nonwoven comprising a first fiber layer comprising a first core/sheath composite fiber having three-dimensional crimp, wherein a sheath component of the first core/sheath composite fiber comprises linear low density polyethylene, a core component of the first core/sheath composite fiber comprises thermoplastic resin having a melting point of at least about 20° C. higher than a melting point of the linear low density polyethylene, and the center of gravity of the core component is offset from the center of gravity of the fiber; a second fiber layer comprising a second core/sheath composite fiber having three-dimensional crimp, wherein a sheath component of the second core/sheath composite fiber comprises high density polyethylene, a core component of the second core/sheath composite fiber comprises thermoplastic resin having a melting point of at least about 20° C. higher than a melting point of the high density polyethylene, and the center of gravity of the core component is offset from the center of gravity of the fiber; wherein at least a portion of the first and the second core/sheath composite fibers is thermally bonded via the sheath components of the first and the second core/sheath composite fibers.

Additionally, the present invention also provides a method for manufacturing a nonwoven comprising forming a first fibrous web comprising a first core/sheath composite fiber having three-dimensional crimp, wherein a sheath component of the first core/sheath composite fiber comprises linear low density polyethylene, a core component of the first core/sheath composite fiber comprises thermoplastic resin having a melting point of at least about 20° C. higher than a melting point of the linear low density polyethylene, and the center of gravity of the core component is offset from the center of gravity of the fiber; forming a second fibrous web comprising a second core/sheath composite fiber having three-dimensional crimp, wherein a sheath component of the second core/sheath composite fiber comprises high density polyethylene, a core component of the second core/sheath composite fiber comprises thermoplastic resin having a melting point of at least about 20° C. higher than a melting point of the high density polyethylene, and the center of gravity of the core component is offset from the center of gravity of the fiber; forming a complex fibrous web by laminating the first fibrous web and the second fibrous web; and subjecting the complex fibrous web to thermal treatment in order to thermally bond at least a portion of the fibers via the sheath components of the first core/sheath composite fiber and the second core/sheath composite fiber.

Furthermore, the present invention also provides a sheet for an absorbent article comprising a nonwoven according to the present invention.

The present invention also provides an absorbent article comprising a topsheet and a backsheet joined to the topsheet, wherein the topsheet comprises the sheet according to the present invention.

These and other features, aspects, and advantages of the present invention will become evident to those skilled in the art from a reading of the present disclosure.

### BRIEF DESCRIPTION OF THE DRAWINGS

FIG. 1 illustrates a fiber cross-section of an example of a core/sheath composite fiber for the nonwoven according to the present invention.

FIGS. 2A to 2C each illustrates a crimping form of a core/sheath composite fiber having three-dimensional crimp.

FIG. 3 illustrates a form of mechanical crimping.

FIG. 4 illustrates another example of a crimping form of a core/sheath composite fiber having three-dimensional crimp.

FIG. 5 is an electronic microscope image of a cross-section of the nonwoven of Example 1.

FIG. 6 is an electronic microscope image of the surface of the first fiber layer of the nonwoven of Example 1.

FIG. 7 is an electronic microscope image of the surface of the second fiber layer of the nonwoven of Example 1.

FIG. 8 is an electronic microscope image of a cross-section of the nonwoven of Comparative Example 1.

FIG. 9 is an electronic microscope image of the surface of the first fiber layer of the nonwoven of Comparative Example 1.

FIG. 10 is an electronic microscope image of the surface of the second fiber layer of the nonwoven of Comparative Example 1.

FIG. 11 is an electronic microscope image of a cross-section of the nonwoven of Comparative Example 4.

### DETAILED DESCRIPTION OF THE INVENTION

All ranges are inclusive and combinable. The number of significant digits conveys neither limitations on the indicated amounts nor on the accuracy of the measurements. All numerical amounts are understood to be modified by the word “about” unless otherwise specifically indicated.

As used herein, absorbent articles include disposable diapers, sanitary napkins, panty liners, incontinence pads, interlabial pads, breast-milk pads, sweat sheets, animal-use excreta handling articles, animal-use diapers, and the like.

The term “joined”, as used herein, refers to the condition where a first member is attached, or connected, to a second member either directly or indirectly. Where the first member is attached, or connected, to an intermediate member which in turn is attached, or connected, to the second member, the first member and second member are joined indirectly.

A nonwoven of the present invention has a laminated structure comprising a first fiber layer and a second fiber layer wherein the first fiber layer and the second fiber layer comprise a first core/sheath composite fiber having three-dimensional crimp and a second core/sheath composite fiber having three-dimensional crimp, respectively and, in the nonwoven, at least a portion of the fibers is thermally bonded via the sheath components of the first core/sheath composite fiber and the second core/sheath composite fiber. A nonwoven of the present invention comprises two types of core/sheath composite fiber with differing sheath components. Without being bound by theory, the sheath component of the first core/sheath composite fiber comprising linear low density polyethylene may impart a luxurious tactile sensation such as surface softness and smoothness. The sheath component of the second core/sheath composite fiber comprising high density polyethylene mainly imparts high bulkiness and cushioning to the nonwoven.

Hereinafter, the fiber constituting the nonwoven of the present invention, the configurations of the first and the second fiber layer, and a method for manufacturing the nonwoven, a sheet from the nonwoven and an absorbent article having the sheet are described.

### First Core/Sheath Composite Fiber

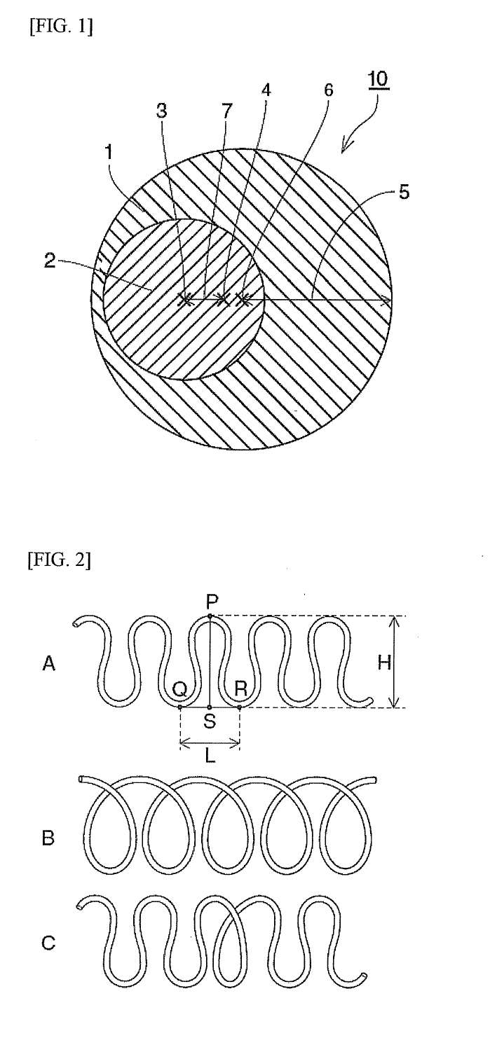

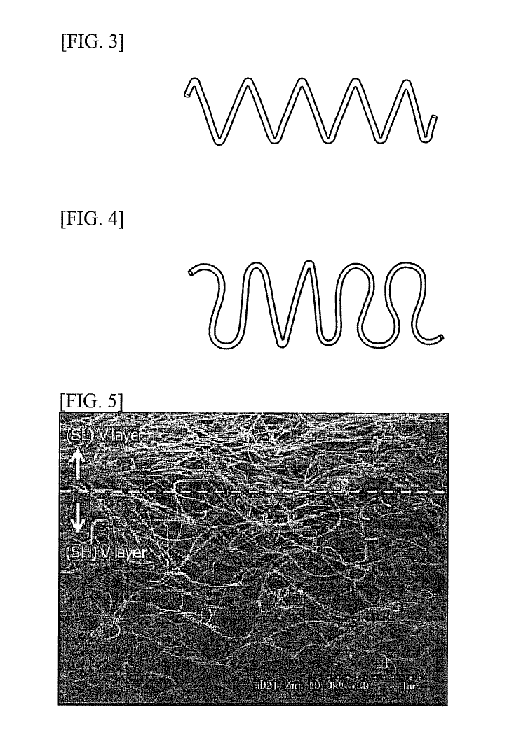

The sheath component of the first core/sheath composite fiber wherein the sheath component thereof comprises linear low density polyethylene and the core component thereof comprises thermoplastic resin having a melting point of at least about 20° C. higher than a melting point of the linear low density polyethylene. In the first core/sheath composite fiber, the center of gravity of the core component is offset from the center of gravity of the fiber. Further, the first core/sheath composite fiber has three-dimensional crimp. Herein, the term “three-dimensional crimp” is used to distinguish from mechanical crimping in which the peaks of the crimped fiber are sharply angled such as those illustrated in FIG. 3. Three-dimensional crimp may refer to crimp where the peaks are curved (wave shaped crimping) as illustrated in FIG. 2A, crimp where the peaks are spiral (spiral shaped crimping) as illustrated in FIG. 2B, crimp where both wave shaped crimping and spiral shaped crimping exist as illustrated in FIG. 2C, or crimp where both mechanical crimp and at least one of wave and spiral shape crimps exist.

The first core/sheath composite fiber is generally provided as an actualized crimping composite fiber. The term “actualized crimping composite fiber” refers to fibers in which three-dimensional crimp is actualized at the fiber stage. The actualized crimping composite fiber differs from a latent crimping composite fiber that develops three-dimensional crimps by thermal treatment involving shrinkage of the fiber. The first core/sheath composite fiber where the center of gravity of the core component is offset from the center of gravity of the fiber is generally provided as an actualized crimping composite fiber.

In the first core/sheath composite fiber, a composite ratio, that is, a ratio of core component/sheath component, is preferably from about 80/20 to about 30/70 (volume ratio), more preferably from about 70/30 to about 35/65, and most preferably from about 60/40 to about 40/60. Without being bound by theory, in the first core/sheath composite fiber, the core component may principally contribute to the bulkiness and the bulkiness recovery characteristics of the nonwoven, and the sheath component may principally contribute to the nonwoven strength and softness of the nonwoven. When the composite ratio is from about 80/20 to about 30/70, both excellent strength and softness of the nonwoven and bulkiness recovery characteristics may be achieved. If the sheath component is increased, the strength of the nonwoven may increase, but the resulting nonwoven may harden and bulkiness recovery characteristics may be compromised. On the other hand, if the core component is excessive, there may be insufficient bonding points, the strength of the nonwoven may decrease and, as a result, bulkiness recovery characteristics may be negatively affected.

In the first core/sheath composite fiber, the center of gravity of the core component is offset from the center of gravity of the fiber in a fiber cross-section which enables explicit crimping characteristics. FIG. 1 illustrates a fiber cross-section of an example of the first core/sheath composite fiber. The sheath component (1) is disposed around the core component (2). As a result, the surface of the sheath component (1) is fused or softened when thermal bonding is conducted. In the fiber cross-section, the center of gravity (3) of the core component (2) is offset from the center of gravity (4) of the fiber (10). In general, the center of gravity (4) of the fiber (10) does not coincide with the center (6) of the fiber (10) since the density of the core component (2) is generally different from the density of the sheath component (1). A degree of shift which may be called an “eccentric ratio” hereinafter, refers to a value obtained from the following equation, in which C1 represents the center of gravity (3) of the core component (2) in the fiber cross-section, Cf represents the center of gravity (4) of the fiber (10), and rf represents a radius of the fiber (10) in the cross-section of the fiber (10). An electron micrograph may be used for determining C1, Cf and rf.

Eccentric ratio (%)[|Cf−C1|/rf]×100

In this equation, |Cf−C1| means a distance between the center of gravity (3) of the core component (2) (that is, the point represented by C1) and the center of gravity (4) of the fiber (10) (that is, the point represented by Cf).

The eccentric ratio of the first core/sheath composite fiber is preferably from about 5% to about 50%, and more preferably from about 7% to about 30% to actualize sufficient three-dimensional crimps without compromising nonwoven productivity, and thereby give a uniform nonwoven with good productivity.

Sheath Component The sheath component of the first core/sheath composite fiber comprises a linear low density polyethylene. The content of linear low density in the sheath component is preferably at least about 60 mass %, and more preferably at least about 75 mass % by mass of the sheath component. The sheath component may comprise only the linear low density polyethylene as a polymer component.

Linear low density polyethylene refers to a copolymer obtained by copolymerizing ethylene and α-olefin. The α-olefin typically has from 3 to 12 carbons. Examples of the α-olefin having from 3 to 12 carbons include propylene, butene-1, pentene-1, 4-methylpentene-1, hexene-1, heptene-1, octene-1, nonene-1, decene-1, dodecene-1, and mixtures thereof. Of these, propylene, butene-1, 4-methylpentene-1, hexene-1, 4-methylhexene-1, and octene-1 are particularly preferable, and butene-1 and hexene are further preferable. The content of the α-olefin in the linear low density polyethylene is preferably from about 1 mol % to about 10 mol % and more preferably from about 2 mol % to about 5 mol %. If the content of the α-olefin is too small, the flexibility of the fiber may be impaired. If the content of the α-olefin is too great, crystallinity may be poor and fibers may become fused together when forming the fiber.

The linear low density polyethylene used in the sheath component may have a density of, for example, from about 0.900 g/cm³ to about 0.940 g/cm³, preferably from about 0.905 g/cm³ to about 0.935 g/cm³, more preferably from about 0.910 g/cm³ to about 0.935 g/cm³ even more preferably from about 0.913 g/cm³ to about 0.933 g/cm³. If the density is less than 0.900 g/cm³, the sheath component may too soften, and sufficient bulkiness and bulkiness recovery characteristics may not be able to be obtained when formed into a nonwoven. In addition, the sheath component may be inferior in terms of rapid cardability. On the other hand, if the density of the linear low density polyethylene is greater than 0.940 g/cm³, when formed into a nonwoven, the surface tactile sensation and softness in the thickness direction of the nonwoven may tend to be inferior.

A melting point of the linear low density polyethylene is preferably within a range of from about 110° C. to about 125° C. If the melting point of the linear low density polyethylene is too high, when manufacturing a nonwoven via thermal bonding at a low temperature, it may not be possible to obtain a nonwoven having a strength that can endure practical use. If the melting point of the linear low density polyethylene is too low, when manufacturing a nonwoven via thermal bonding at a high temperature, the surface tactile sensation of the nonwoven may decline, or the rapid cardability during nonwoven manufacturing may be inferior and the obtained nonwoven may not have an excellent uniformity.

The linear low density polyethylene for the present invention can be easily obtained by copolymerizing ethylene with α-olefin using a metallocene catalyst. Moreover, the linear low density polyethylene is not limited to a product of polymerization using a metallocene catalyst and, may be a product obtained via polymerization using a Ziegler-Natta catalyst.

The linear low density polyethylene used in the sheath component preferably has a melt index (MI) in the range of from 1 g/10 min to 60 g/min considering the spinnability, more preferably from 2 g/10 min to 40 g/10 min, even more preferably from 3 g/10 min to 35 g/10 min, and most preferably 5 g/10 min to 30 g/min. The MI is determined in accordance with JIS-K-7210 (1999) (Conditions: 190° C., load 21.18 N (2.16 kgf)). As the MI is larger, the solidification speed of the sheath component is slower, resulting in fusion of fibers. On the other hand, when MI is too low, the fiber production tends to be difficult.

A ratio (Q value: Mw/Mn) of the weight average molecular weight (Mw) to the number-average molecular weight (Mn) of the linear low density polyethylene is preferably not more than about 5. The Q value is more preferably from about 2 to about 4, and even more preferably from about 2.5 to about 3.5. The Q value of not greater than 5 means that the breadth of the molecular weight distribution of the linear low density polyethylene is narrow. A composite fiber with superior explicit crimping properties can be obtained by using the linear low density polyethylene with a Q value within the range described above as the sheath component.

From the perspectives of the characteristics of the resulting composite fiber, and the tactile sensation and bulkiness of a fiber aggregate using the composite fiber, a flexural modulus of the linear low density polyethylene is preferably within a range of about 65 MPa to about 850 MPa, more preferably from about 120 MPa to about 750 MPa, even more preferably from about 180 MPa to about 700 MPa, and most preferably from about 250 MPa to about 650 MPa. Herein, “flexural modulus” is measured in accordance with Japanese Industrial Standards (“JIS”) K 7171 (2008). The first core/sheath composite fiber comprising the linear low density polyethylene as a main component of the sheath has a pliable tactile sensation. However, without a certain degree of firmness the fiber may result in a decrease in carding performance, and may also make it difficult to obtain a fiber aggregate having high bulkiness and high resiliency. As such, the linear low density polyethylene preferably has a degree of deformation resistance with respect to flexing (that is, preferably has a somewhat high degree of deformation resistance with respect to flexing), and preferably has a flexural modulus of at least about 65 MPa. If the flexural modulus of the linear low density polyethylene is too high, the pliable tactile sensation of obtained nonwoven may be deteriorated.

From the perspectives of the characteristics of the resulting composite fiber, and the tactile sensation, bulkiness, and resiliency of a fiber aggregate using the composite fiber, a hardness of the linear low density polyethylene is preferably in a range of from about 45 to about 75, more preferably from about 48 to about 70, even more preferably from about 50 to about 65, and most preferably from about 50 to about 62. Herein, the “hardness of the linear low density polyethylene” refers to durometer hardness (HDD) measured using a type-D durometer in accordance with JIS K 7215 (1986). If the linear low density polyethylene is too soft, the firmness of the fiber may be lost, the carding performance of the fiber may decline, and it may become difficult to obtain a bulky fiber aggregate. Moreover, the bulkiness recovery characteristics of the fiber aggregate may also decrease. If the hardness of the linear low density polyethylene is too high, there is a possibility that the pliable tactile sensation of a resultant nonwoven may be deteriorated.

Provided that three-dimensional crimp is sufficiently actualized in the first core/sheath composite fiber and that the resultant nonwoven gives good tactile sensation, the sheath component may further comprise polymer components other than the linear low density polyethylene. For example, the sheath component may further comprise, as an additional polymer, one or more types of polymers selected from a group consisting of a polyolefin-based resin such as high density polyethylene, branched low density polyethylene, polypropylene, polybutene, polybutylene, polymethylpentene resin, polybutadiene, propylene-based copolymers (e.g. propylene-ethylene copolymer), ethylene-vinyl alcohol copolymer, ethylene-vinyl acetate copolymer, ethylene-(meth)acrylate copolymer, or ethylene-(meth)acrylate methyl copolymer, and the like; polyester resins such as polyethylene terephthalate, polybutylene terephthalate, polytrimethylene terephthalate, polyethylene naphthalate, polylactic acid, polybutylene succinate, and copolymers thereof; polyamide-based resins such as nylon 66, nylon 12, nylon 6, and the like; acrylic resin; engineering plastics such as polycarbonate, polyacetal, polystyrene, cyclic polyolefin, and the like; mixtures thereof; and elastomer-based resins thereof.

As the additional polymer, branched low density polyethylene is preferable in respect with actualization and stabilization of three-dimensional crimp without compromising surface softness and smoothness. Further, branched low density polyethylene can serves as a “softener” to the linear low density polyethylene and can provide softness in the thickness direction of a nonwoven. By adding branched low density polyethylene, it is possible to process nonwoven in a wide range of temperature, therefore, when nonwoven is thermally-bonded, nonwoven having uniform softness can be obtained regardless nonwoven process temperature. The branched low density polyethylene used in the sheath component may have a density of, for example, from about 0.910 g/cm³ to about 0.930 g/cm³. The branched low density polyethylene has a melting point which is lower, preferably at least 5° C. lower and more preferably 10° C. lower than a melting point of the linear low density polyethylene.

The branched low density polyethylene used in the sheath component preferably has a melt index (MI) in the range of from 1 g/10 min to 60 g/min considering the spinnability, more preferably from 3 g/10 min to 50 g/10 min, even more preferably from 5 g/10 min to 50 g/10 min, and most preferably 10 g/10 min to 50 g/min. The MI is determined in accordance with JIS-K-7210 (1999) (Conditions: 190° C., load 21.18 N (2.16 kgf)). As the MI is larger, the solidification speed of the sheath component is slower, resulting in fusion of fibers. On the other hand, when MI is too low, the fiber production tends to be difficult.

In one embodiment, linear low density polyethylene and branched low density polyethylene preferably accounts for about 70 mass %, more preferably about 80% and even more preferably about 90 mass % of the sheath component. In such embodiment, linear low density polyethylene preferably accounts for about 95 mass % to about 75 mass % and more preferably about 90 mass % to about 80 mass % of the total mass of linear low density polyethylene and branched low density polyethylene.

The sheath component may comprise additives other than the polymer component, such as anti-static agents, pigments, matting agents, thermal stabilizers, light stabilizers, flame retardants, antimicrobial agents, lubricants, plasticizers, softeners, antioxidants, ultraviolet absorbers, crystal nucleating agents, and the like. These additives are preferably included in the sheath component at an amount that is not more than about 10 mass % of the entire sheath component.

Core Component

The core component comprises thermoplastic resin having a melting point that is at least about 20° C. higher than a melting point of the linear low density polyethylene in the sheath component of the first core/sheath composite fiber as a polymer component, preferably in an amount of at least about 50 mass % and more preferably at least about 75 mass %, of by mass of the core component. The thermoplastic resin preferably includes a polyolefin-based resin such as polypropylene, polymethylpentene, and the like; polyester resins such as polyethylene terephthalate, polybutylene terephthalate, polytrimethylene terephthalate, polyethylene naphthalate, polylactic acid, and copolymers thereof; polyamide-based resins such as nylon 66, nylon 12, nylon 6, and the like; acrylic resin; engineering plastics such as polycarbonate, polyacetal, polystyrene, cyclic polyolefin, and the like; mixtures thereof. For the perspectives of the uniformity of the nonwoven and nonwoven productivity, polyolefin resin, polyester and polyamide-based resin are more preferable. Examples of the polyester include polymers and copolymers such as polyethylene terephthalate, polybutylene terephthalate, polytrimethylene terephthalate, polyethylene naphthalate, polylactic acid. Polyethylene terephthalate and polybutylene terephthalate are preferred, and polyethylene terephthalate are more preferred. A melting point of the polyester is preferably at least about 40° C. higher, more preferably at least 50° C. higher, than the melting point of the linear low density polyethylene of the sheath component. Alternatively, the core component may comprise only the polyester as a polymer component.

The core component may comprise additives other than the polymer component, such as anti-static agents, pigments, matting agents, thermal stabilizers, light stabilizers, flame retardants, antimicrobial agents, lubricants, plasticizers, softeners, antioxidants, ultraviolet absorbers, crystal nucleating agents, and the like. These additives are preferably included in the core component at an amount that is not more than about 10 mass % of the core component.

### Second Core/Sheath Composite Fiber

The sheath component of the second core/sheath composite fiber comprises high density polyethylene and the core component thereof comprises thermoplastic resin having a melting point which is at least about 20° C. higher than a melting point of the high density polyethylene. The center of gravity of the core component is offset from the center of gravity of the fiber. Furthermore, the second core/sheath composite fiber has three-dimensional crimp. “Three-dimensional crimp” has the same meaning as that described in connection with the first core/sheath composite fiber. The second core/sheath fiber is generally provided as an actualized crimping composite fiber. The preferable composite ratio and the preferable eccentric ratio of the second core/sheath composite fiber are as described in connection with the first core/sheath composite fiber. The cross-section of the second core/sheath composite fiber is also as described in connection with the first core/sheath composite fiber.

Sheath Component

The sheath component of the second core/sheath composite fiber comprises high density polyethylene, preferably in an amount of at least 60 mass %, and more preferably at least about 75 mass %, of by the mass of the sheath component. Alternatively, the sheath component may comprise only the high density polyethylene as a polymer component. The high density polyethylene is a hard polyethylene with little branching. It is also referred to as low-pressure polyethylene as it is produced via a low-pressure process. Without being bound by theory, the second core/sheath composite fiber with high density polyethylene may impart increased bulkiness and the cushioning to the nonwoven.

A density of the high density polyethylene is preferably from about 0.940 g/cm³ to about 0.970 g/cm³, and more preferably from about 0.945 g/cm³ to about 0.960 g/cm³ to actualize sufficient three-dimensional crimp without compromising nonwoven productivity.

A melting point of the high density polyethylene is preferably from about 120° C. to about 140° C., more preferably from about 123° C. to about 138° C. and even more preferably from about 125° C. to about 135° C. By having the melting point within this range, it is possible to avoid decrease of a thickness of a fiber web comprising the second core/sheath composite fiber in the nonwoven manufacturing process according to the present invention. Preferably, a melting point of the high density polyethylene of the second core/sheath composite fiber is higher than that of the linear low density polyethylene of the first core/sheath composite fiber for the perspective of securing bulkiness and resiliency of a nonwoven. In one embodiment, a melting point of the high density polyethylene of the second core/sheath composite fiber is higher at least 3° C., preferably 5° C., more preferably 8° C. than that of the linear low density polyethylene of the first core/sheath composite fiber.

Provided that three-dimensional crimp is sufficiently actualized in the second core/sheath component may comprise polymer components other than the high density polyethylene. The other polymer components that the sheath component may comprise are the same as the other components that the sheath component of the first core/sheath composite fiber may comprise, as described above except for high density polyethylene. Alternatively, the sheath component may comprise linear low density polyethylene as a polymer component.

The sheath component may comprise an additive in addition to the polymer component. The additives are the same as the additives that the sheath component of the first core/sheath composite fiber may comprise, as described above. These additives are preferably included in the sheath component at an amount that is not more than about 10 mass % of the entire sheath component.

The high density polyethylene used in the sheath component preferably has a melt index (MI) in the range of from 3 g/10 min to 50 g/min considering the spinnability, more preferably from 5 g/10 min to 50 g/10 min, even more preferably from 7 g/10 min to 40 g/10 min, and most preferably 8 g/10 min to 30 g/min. The MI is determined in accordance with JIS-K-7210 (1999) (Conditions: 190° C., load 21.18 N (2.16 kgf)). As the MI is larger, the solidification speed of the sheath component is slower, resulting in fusion of fibers. On the other hand, when MI is too low, the fiber production tends to be difficult.

Core Component

The core component of the second core/sheath composite fiber comprises thermoplastic resin having a melting point which is at least about 20° C. higher than a melting point of the high density polyethylene in the sheath component of the second core/sheath composite fiber as a polymer component, preferably in an amount of at least about 50 mass % of by mass of the core component. Alternatively, the core component may comprise only the polyester as a polymer component.

Descriptions for preferred thermoplastic resin provided for a core component of the first core/sheath composite fiber except that a melting point of polyester for a core component is preferably at least about 40° C. higher, more preferably at least 50° C. higher, than the melting point of the high density polyethylene of the sheath component of the second core/sheath composite fiber, are also applicable for thermoplastic resin as a core component of the second core/sheath composite fiber.

Three-Dimensional Crimp in the First and the Second Core/Sheath Composite Fibers

In both the first and the second core/sheath composite fibers, the number of three-dimensional crimp is preferably from about 6 to about 26 crimps/25 mm, and more preferably about 8 to about 22 crimps/25 mm from the perspective of bulkiness and cushioning when the fiber is formed into a nonwoven as well as nonwoven productivity. If less than 6 crimps/25 mm are provided, carding performance may decline and the bulkiness and bulkiness recovery characteristics of the nonwoven may not be secured. If more than 26 crimps/25 mm are provided, carding performance and the uniformity of the nonwoven may negatively be affected.

Additionally, when measured in accordance with JIS L 1015 (2010), a crimping rate is preferably from about 5% to about 25%, and more preferably from about 8% to about 23% from the perspective of good carding performance of the fiber as well as high bulkiness and cushioning properties of the resulting nonwoven. A ratio of the crimping rate to the number of crimps (crimping rate/number of crimps) is preferably from about 0.4 to about 1.2 and more preferably from about 0.5 to about 1. Without being bound by theory the crimping rate may be an indication of the fixedness of the crimping (resistance to stretching of the crimps). When crimping rate/number of crimps is in the range above, the crimps may not easily stretch and the fiber will have three-dimensional crimp of an appropriate size. As results, excellent nonwoven productivity, and bulkiness and resiliency of the obtained nonwoven can be achieved.

In both the first and the second core/sheath composite fibers, the fineness of the fiber are not particularly limited. For example, the fiber can be a short fiber having a fineness of about 1.1 dtex to about 15 dtex, preferably about 1.5 dtex to about 5 dtex. A fiber length is preferably in the range of about 1 mm to about 100 mm, more preferably about 28 mm to about 72 mm, and even more preferably about 32 mm to about 64 mm for producing a card web in cases where a fiber web such as a card web is produced using a carding machine when fabricating a nonwoven. In a case using an air-laid machine, a fiber length is preferably in the range of from about 3 mm to about 30 mm and more preferably in the range of from about 5 mm to about 25 mm. The fineness of the fiber can be adjusted as desired by adjusting the fineness of the spun filament and the stretch factor. A fiber having a predetermined length can be obtained by cutting the fiber after the annealing. In one embodiment, a fiber length of a first core/sheath composite fiber is shorter than that of a second core/sheath composite fiber for the perspective of surface smooth and softness of a nonwoven. In the embodiment, a fiber length of a first core/sheath composite fiber is preferably in the range of about 28 mm to about 60 mm and more preferably about 28 mm to about 51 mm, and a fiber length of a second core/sheath composite fiber is preferably in the range of about 32 mm to about 70 mm and more preferably about 40 mm to about 64 mm.

### Manufacturing the First and Second Core/Sheath Composite Fibers

Both the first and the second core/sheath composite fibers can be manufactured according to the following procedure. First, a sheath component comprising a predetermined amount of polyethylene and a core component comprising a predetermined amount of thermoplastic resin (for example, polyester) are melt-spun using an eccentric core/sheath composite nozzle. A spinning temperature of the core component is, for example, from about 240° C. to about 350° C., a spinning temperature of the sheath component is, for example, from about 200° C. to about 300° C., and a pulling speed is from about 100 m/min to about 1500 m/min. Thus, a spun filament is obtained.

Next, the spun filament is subjected to drawing processing at a stretch factor of at least about 1.5 times. A drawing temperature is at least the glass transition temperature (Tg₂) of the polymer components included in the core component having the highest glass transition temperature and less than the melt peak temperature of the polyethylene included in the sheath component. The lower limit of the drawing temperature is more preferably a temperature which is 10° C. higher than Tg₂. The upper limit of the drawing temperature is more preferably 90° C., and even more preferably 85° C. If the drawing temperature is lower than Tg₂, progress of the crystallization of the core component may be inhibited and, as a result, the thermal shrinkage of the core component in the resulting fiber may tend to increase, or the bulkiness and/or recovery characteristics of the nonwoven produced using the resulting fiber may tend to decline. It is not preferable that the drawing temperature is greater than the melt peak temperature of the polyethylene (linear low density polyethylene for the first core/sheath composite fiber, high density polyethylene for the second core/sheath composite fiber) because the fibers may fuse.

To obtain a fiber that actualizes wave shaped crimping and/or spiral shaped crimping, an appropriate stretch factor is necessary. The lower limit of the stretch factor is more preferably 1.8 times, even more preferably 2.0 times, and most preferably 2.2 times. The upper limit of the stretch factor is more preferably 5.0 times, even more preferably 4.0 times, and most preferably 3.8 times. If the stretch factor is less than 1.5 times, the stretch factor will be too low and it will be difficult to obtain a fiber that actualizes wave shaped crimping and/or spiral shaped crimping. Additionally, not only will the bulkiness when formed into a nonwoven be reduced, but the rigidity of the fiber itself will decline, leading to a tendency for nonwoven productivity (e.g. carding performance and the like) to decline or, alternatively, bulkiness recovery characteristics to decline. Additionally, as needed, the resulting filament may be subjected to annealing in a dry heat, wet heat, or steam heat atmosphere at a temperature at which the fibers do not fuse, from 50° C. to 115° C., before or after the stretching.

Next, as needed, before or after adding the fiber treatment agents, from 6 crimps/25 mm to 26 crimps/25 mm are provided to the fiber using a conventionally known crimping apparatus such as a stuffing box crimper. The shape of the crimps, after the fiber passes through the crimper, may be sawtooth shaped and/or wave shaped.

Furthermore, after crimping using the crimping apparatus described above, the fiber is preferably subjected to annealing in a dry heat, wet heat, or steam heat atmosphere at a temperature from about 50° C. to about 115° C. The actualization of the three-dimensional crimp in the fiber can be promoted by the annealing. Specifically, it is preferable to perform the crimping using the crimping apparatus after adding the fiber treatment agents, and then to perform annealing and drying at the same time in a dry heat atmosphere at a temperature from 50° C. to 115° C. because the procedure can be simplified. If the annealing temperature is less than 50° C., the dry heat shrinkage ratio of the resulting fiber may tend to increase, and thereby, the texture of the resulting nonwoven may be deteriorated, and productivity may decline. Additionally, in cases where performing the annealing step and the drying step simultaneously, if the annealing temperature is less than 50° C., the drying of the fiber may be insufficient. Through the method described above, fibers actualizing three-dimensional crimp can be obtained.

The first fiber layer may differ from the second fiber layer in hydrophilicity. When the nonwoven according to the present invention is to be used as a topsheet in an absorbent article, it is desirable that the first fiber layer is less hydrophilic than the second fiber layer. For example, the first and the second core/sheath composite fibers can be treated with a treatment agent such as a hydrophilic agent, rendering the first core/sheath composite fiber less hydrophilic than the second core/sheath composite fiber. Such hydrophilic agents may for example include or be a surfactant. The first fiber layer can be less hydrophilic than the second fiber layer by treating the first core/sheath composite fiber with less hydrophilic treatment agent than one treating the second core/sheath composite fiber, or by treating the first core/sheath composite fiber with a hydrophilic treatment agent which can be more easily removed from the fiber. By rendering the first fiber layer less hydrophilic than the second fiber layer, when a nonwoven according to the present invention is used as a topsheet of an absorbent article in a way that the surface of the first fiber layer faces the skin, the surface of the topsheet can maintain enhanced dryness.

### Configuration of Nonwoven

The nonwoven of the present invention comprises a first fiber layer comprising the first core/sheath composite fiber and a second fiber layer comprising the second core/sheath composite fiber. At least a portion of the fibers is thermally bonded via the sheath components of these two types of core/sheath composite fibers.

The first fiber layer comprises preferably at least about 50 mass %, more preferably at least about 70 mass %, and even more preferably at least about 80 mass % of the first core/sheath composite fiber. Moreover, the first fiber layer may be constituted by only the first core/sheath composite fiber.

The second fiber layer comprises preferably at least about 50 mass %, more preferably at least about 70 mass %, and even more preferably at least about 80 mass % of the second core/sheath composite fiber. Moreover, the second fiber layer may be constituted by only the second core/sheath composite fiber.

The first fiber layer and the second fiber layer may include other fibers than the first core/sheath composite fiber and the second core/sheath composite fiber, respectively. Examples of the other fibers include natural fibers such as cotton, silk, wool, hemp, pulp, and the like; reclaimed fiber such as rayon, cupra, and the like; and synthetic fibers such as acrylic-based, polyester-based, polyamide-based, polyolefin-based, and polyurethane-based fibers. One type or a plurality of types can be selected from these fibers, based on the application of the nonwoven.

A basis weight of the first fiber layer and the second fiber layer, respectively, is preferably from about 5 g/m² to about 50 g/m², more preferably from about 10 g/m² to about 40 g/m², and even more preferably from about 14 g/m² to about 35 g/m². A ratio of a basis weight of the first fiber layer/a basis weight of the second fiber layer is preferably from about 70/30 to about 20/80, more preferably from about 60/40 to about 30/70, and even more preferably from about 55/45 to about 35/65. If the basis weight of the first fiber layer is too small and/or the ratio of the basis weight of the first fiber layer to the basis weight of the second fiber layer is too small, excellent tactile sensation may not be obtained or, rather, softness and smoothness in the surface of the first fiber layer may decline. If the basis weight of the first fiber layer is too large and/or the ratio of the basis weight of the first fiber layer to the basis weight of the second fiber layer is too large, the bulkiness and the cushioning of the nonwoven may decline.

In the nonwoven of the present invention, the first fiber layer has preferably a higher fiber density than the second fiber layer. Such difference in fiber density between the first and second fiber layers can result in not only improved surface softness and tactile sensation but also improved dryness feeling and prevention of rewet when the nonwoven is employed as a topsheet in absorbent articles.

A fiber density of a fiber layer may be evaluated from a specific volume of the fiber layer. A smaller specific volume indicates a more compact fiber layer. Alternatively, a fiber density of a fiber layer can be evaluated by observing a predetermined region of cross-section obtained by cutting the nonwoven in the thickness direction and comparing the ratios of voids in the regions (e.g. the ratio of the area of the voids). A smaller ratio of voids in the region can be understood to indicate higher fiber density.

One possible way to obtain the first fiber layer having a higher fiber density than the second fiber layer may be configuring the intensity (degree) of the three-dimensional crimp of the first core/sheath composite fiber included in the first fiber layer to be less than that of the second core/sheath composite fiber included in the second fiber layer. The intensity of the three-dimensional crimp may be evaluated by a ratio of the height (“H” in FIG. 2A) of the peaks of the three-dimensional crimp, i.e. a distance between the apex (“P” in FIG. 2) of the peak and the bottom (“S” in FIG. 2) of the valley, to the distance (“L” in FIG. 2A) between bottoms (“Q” and “R” in FIG. 2A) of two adjacent valleys of the three-dimensional crimp. It also can be evaluated by the number of crimps measured in accordance with JIS L 1015 (2010). Greater heights of the peaks, smaller spacing between two adjacent valleys, and higher numbers of crimps indicate more intense three-dimensional crimp.

Alternatively, or in addition thereto, as described hereinafter, the first fiber layer having a higher fiber density than the second fiber layer may be obtained by bringing the fiber web that becomes the first fiber layer in contact with a conveying support (e.g. conveyor belt) of the thermal treatment apparatus, in the thermal treatment performed when manufacturing the nonwoven. If the first fiber layer is in contact with the conveying support during the thermal treatment, the first fiber layer will be pressed on by support and, as a result, it will be easier to make the fiber layer more compact and the surface of the fiber layer will be more smoother. Therefore, a smoother tactile sensation will be imparted to the surface of the nonwoven.

In the nonwoven of the present invention, the L/H of the first core/sheath composite fiber included in the first fiber layer is prone to become greater than the L/H of the second core/sheath composite fiber included in the second fiber layer. This is thought to be due to the linear low density polyethylene included in the sheath component of the first core/sheath composite fiber having a melting point that is lower than that of the high density polyethylene included in the sheath component of the second core/sheath composite fiber. That is, this is thought to be a result of the shape of the three-dimensional crimp being easily lost when thermal treating the fibrous web, because of increased deformation caused by softening and melting in the first core/sheath composite fiber, which leads to the easy flattening of the first core/sheath composite fiber. The L/H of the first fiber layer increases when the flattening of the first core/sheath composite fiber increases, and this leads to an increase in the difference with the L/H of the second core/sheath composite fiber. In the case where the L/H of the first core/sheath composite fiber included in the first fiber layer is large, this means that the three-dimensional crimp in the first core/sheath composite fiber weakens due to the thermal treatment, and the shape of the fiber becomes flatter. As a result, the tactile sensation experienced when stroking the surface of the first fiber layer will be smooth. On the other hand, the crimped shape in the second fiber layer is quite maintained even when thermal treatment is performed and, thus, the second fiber layer has greater bulk. Therefore, when the nonwoven of the present invention is used in a topsheet for an absorbent article in which the first fiber layer is arranged as the surface in contact with the skin, both a smooth tactile sensation and a feathery overall bulkiness of the topsheet can be achieved.

In the nonwoven of the present invention, a ratio of the L/H of the first core/sheath composite fiber (hereinafter L1/H1) to the L/H of the second core/sheath composite fiber (hereinafter L2/H2) (i.e. (L1/H1)/(L2/H2)) is preferably at least 1.05. When the ratio of the L/H of the first core/sheath composite fiber to the L/H of the second core/sheath composite fiber is 1.05 or greater, the tactile sensation of the first fiber layer will be smooth and the nonwoven will be bulky and feathery. If the ratio of the L/H of the first core/sheath composite fiber to the L/H of the second core/sheath composite fiber is less than 1.05, such a configuration will result in the first fiber layer not being able to display a smooth tactile sensation and/or the bulkiness of the second fiber layer being reduced, which leads to the nonwoven becoming thin and the feathery sensation not being possible to attain. The ratio of the L/H of the first core/sheath composite fiber to the L/H of the second core/sheath composite fiber is more preferably at least 1.1, even more preferably at least 1.15, and most preferably at least 1.2. The upper limit of the ratio of the L/H of the first core/sheath composite fiber to the L/H of the second core/sheath composite fiber is not particularly limited, but preferably is about 3 or less, more preferably 2.5 or less, and even more preferably 2 or less.

A basis weight of the nonwoven may be appropriately selected depending on the nonwoven application. For the nonwoven of the present invention as a topsheet of an absorbent article, the integral basis weight of the first fiber layer and the second fiber layer of the nonwoven is preferably from about 28 g/m² to about 70 g/m², more preferably about 35 g/m² to about 65 g/m². For the use of the nonwoven as a topsheet, in one embodiment, when the integral basis weight of the nonwoven is in the range of from about 47 g/m² to about 70 g/m², the basis weight of the first fiber layer is preferably 20%-70%, more preferably, 30-65% of the integral basis weight. In another embodiment, when the integral basis weight of the nonwoven is in the range of from about 28 g/m² to not greater than 47 g/m², the basis weight of the first fiber layer is preferably 40%-75%, more preferably, 50-70% of the integral basis weight.

In one embodiment, the nonwoven may be constituted by only the first fiber layer and the second fiber layer. In another embodiment, the nonwoven comprises three layers in which the first fiber layer is layered on both faces of the second fiber layer. In another embodiment, the nonwoven may include at least one additional fiber layer in addition to the first and second fiber layers. The fiber for the additional fiber layer can be selected from natural fibers such as cotton, silk, wool, hemp, pulp, and the like; reclaimed fibers such as rayon, cupra, and the like; and synthetic fibers such as acrylic-based, polyester-based, polyamide-based, polyolefin-based, and polyurethane-based fibers. Such additional fiber layer may be constituted by one or more types of fibers selected from these fibers.

### Nonwoven Manufacturing Process

The nonwoven may be manufactured via a process including the steps of: forming a first fibrous web comprising the first core/sheath composite fiber, forming a second fibrous web comprising the second core/sheath composite fiber, forming a complex fibrous web by laminating the first fibrous web and the second fibrous web, and subjecting the complex fibrous web to thermal treatment in order to thermally bond at least a portion of the fibers via the sheath portions of the first core/sheath composite fiber and the second core/sheath composite fiber.

The first fibrous web and the second fibrous web may be carded webs such as parallel webs, semi-random webs, random webs, cross-webs, criss-cross webs, and the like, air-laid webs, wet-laid webs, and spunbond webs, and the like. The first and the second fibrous webs may be the same, or different.

The thermal treatment of a complex fibrous web can be conducted using a conventionally known thermal treatment method. An example of a preferable treating process is one in which a thermal treatment apparatus is used where the fibrous web is not subjected to a great deal of pressure such as air pressure, such as a hot air through-type thermal treatment apparatus, a hot air blowing thermal treatment apparatus, a infrared thermal treatment apparatus, or the like. These thermal treatment apparatuses are typically provided with a conveying support for supporting and conveying a fibrous web. Thermal treatment may be performed under conditions such that the sheath components of the first and the second core/sheath composite fibers sufficiently melt and/or soften, and bond at a point of contact or intersection of the fibers, and such that the three-dimensional crimp of the first and the second core/sheath composite fiber does not collapse. For example, the thermal treatment temperature may be from about 125° C. to about 150° C., and preferably from about 128° C. to about 145° C.

### Application of the Nonwoven

The nonwoven of the present invention delivers a soft and smooth feel to the skin, has a bulky and feathery tactile sensation when the surface of the nonwoven is pressed against, and has an appropriate amount of cushioning and bulkiness recovery characteristics.

As such, the nonwoven of the present invention can be preferably used in applications in which the nonwoven is in contact with the skin, specifically applications in which the first fiber layer is the surface that is in contact with the skin. For example, the nonwoven of the present invention can be used in applications such as products that contact human or non-human animal skin, such as infant-use disposable diapers, adult-use disposable diapers, sanitary napkins, panty liners, incontinence pads, interlabial pads, breast-milk pads, sweat sheets, animal-use excreta handling articles, animal-use diapers, and similar various absorbent articles; face masks, base fabric of cooling/heating pads and similar cosmetic/medical-use patches, wound surface protection sheets, nonwoven bandages, hemorrhoid pads, warming devices that directly contact the skin (e.g. disposable hand warmers), base fabric of various animal-use patches, and similar skin covering sheets; makeup removal sheets, anti-perspirant sheets, bottom wipes and similar wipes for use on a person, various wiping sheets for use on animals, and the like. The nonwoven of the present invention is preferably used as a topsheet for an absorbent article in which the surface of first fiber layer is in contact with the skin.

Absorbent Article

An absorbent article according to the present invention comprises a topsheet; and a backsheet joined to the topsheet, wherein the topsheet comprises the nonwoven according to the present invention. It may further comprise an absorbent core.

The absorbent articles of the present invention may be produced industrially by any suitable means. The different layers may thus be assembled using standard means such as embossing, thermal bonding, or gluing or combination of both.

Topsheet

Topsheet can catch body fluids and/or allow the fluid penetration inside the absorbent article. With the nonwoven according to the present invention, the first fiber layer is preferably, disposed on a side in contact with the skin.

Backsheet

Any conventional liquid impervious backsheet materials commonly used for absorbent articles may be used as backsheet. In some embodiments, the backsheet may be impervious to malodorous gases generated by absorbed bodily discharges, so that the malodors do not escape. The backsheet may or may not be breathable.

Absorbent Core

It may be desirable that the article further comprises an absorbent core disposed between the topsheet and the backsheet. As used herein, the term “absorbent core” refers to a material or combination of materials suitable for absorbing, distributing, and storing fluids such as urine, blood, menses, and other body exudates. Any conventional materials for absorbent core suitable for absorbent articles may be used as absorbent core.

### Test Methods

Measurement of Estimated Compressible Thickness

Estimated compressible thickness of nonwoven is measured using MTS Criterion Model 42 (MTS Systems Corporation) with 2N force. Estimated compressible thickness means the distance that the MTS crosshead travels from force 0.01N (contact) to force 2N (maximum force applied to the sample).

Measurement of Standard Mean Deviation of Surface Roughness (SMD)

The surface tactile sensation of the nonwoven can be measured and evaluated based on the KES (Kawabata Evaluation System), which is a method for measuring the feeling of fabric and conducting an objective evaluation of the same. The surface tactile sensation of the nonwoven can be evaluated by measuring the surface friction property value as defined by the KES. Specifically, the standard mean deviation of surface roughness (hereinafter referred to as “SMD”) is measured as the surface friction property value.

Larger SMD indicates greater unevennesses in the surface. A device used to measure SMD is not particularly limited, provided that it is a device capable of taking measurements of surface friction based on the KES. For example, the surface friction property value can be measured using a KES-SE friction sensitivity tester, a KES-FB4-AUTO-A automatic surface tester (both manufactured by Kato Tech Co., Ltd.), or the like. The surface friction can be measured by applying a static load of 25 gf, and setting the movement speed of the friction block to 1 mm/sec in the vertical direction of the nonwoven as the measurement direction. At least one surface of the nonwoven preferably has SMD of 3.5 or less, more preferably 3.0 or less, and even more preferably 2.5 or less, and most preferably 1.9 or less. The nonwoven surface with SMD of 4.0 or less has less convexities, making the surface tactile of the product smooth. The lower limit of SMD is not particularly limited, preferably close to zero, but may be 0.3 or 0.5.

Measurement of L/H

A scanning electronic microscope image (Hitachi, S3500N-2) of an about 20 mm² nonwoven sample is taken. Magnification is selected from a range of 20× to 100×, generally 30×, such that the surface of the nonwoven was observed sufficiently to measure H and L. The height (“H” in FIG. 2A) of the peaks of the three-dimensional crimp and the distance (“L” in FIG. 2A) between bottoms (“Q” and “R” in FIG. 2A) of two adjacent valleys of wave shape crimp are measured in fibers showing wave-type crimps, and average H and L are obtained from values of H and L from 5 different fibers.

Measurement of Work of Compression

1) KES Method

The softness and resiliency in the thickness direction of the nonwoven can also be measured and evaluated based on the KES by measuring the compression property value defined in the KES, which is derived from the behavior of the load-displacement curve when compression testing.

Of the compression property values defined by the KES, softness in the thickness direction can be evaluated by measuring compression energy (also called “work of compression”; hereinafter referred to as “WC” (gf·cm/cm²)). Larger value of WC indicates greater softness in the thickness direction and greater ease of compression. The compression property value, for example, can be measured using a KES-G5 compression tester, a KES-FB3-AUTO-A automatic compression tester (both manufactured by Kato Tech Co., Ltd.), or the like. In the nonwoven of the present invention, WC is preferably 2.00 gf·cm/cm² or more, more preferably 2.75 gf·cm/cm² or more, most preferably 2.9 gf·cm/cm² or more. The nonwoven with WC of 2.00 gf·cm/cm² or more highly deforms when load is applied thereto, giving more feathery feeling. The upper limit of WC is not particularly limited. If WC is over 8.0 gf·cm/cm², other compression properties may be affected. For this reason, WC is preferably 6.0 gf·cm/cm² or less, more preferably 4.0 gf·cm/cm² or less.

Resilience of compression, hereinafter referred to as “RC” (%), indicates resilience to compression, or recoverability or repulsion. Larger values indicate ease of repulsion to compression, that is, greater cushioning.

In the nonwoven of the present invention, RC is preferably at least about 50%, more preferably at least about 55%, and more preferably at least about 60%. The upper limit of RC is not particularly limited, and may be 100%, 90%, or 85%.

The bulkiness of the nonwoven can be expressed in terms of specific volume. The specific volume is calculated by dividing thickness by basis weight. Note that, however, the specific volume varies based on the storage state of the nonwoven and/or nonwoven manufacturing process. For example, if the nonwoven is wound around a core and stored in a rolled-up state, the nonwoven on the side closer to the core will tend to have a smaller specific volume. For example, the nonwoven of the present invention preferably has a specific volume immediately after manufacture of about 60 cm³/g to about 150 cm³/g, and preferably of about 65 cm³/g to about 130 cm³/g. In another example, when the nonwoven is employed as a topsheet in an absorbent article, the nonwoven topsheet in the absorbent article has a specific volume of preferably about 10 cm³/g to about 60 cm³/g, and more preferably of about 15 cm³/g to about 50 cm³/g, and even more preferably of about 20 cm³/g to about 40 cm³/g.

### EXAMPLES

### Manufacture of Core/Sheath Composite Fiber A-1

Umerit® 631J (Ube-Maruzen Polyethylene Co., Ltd.; density: 0.931 g/cm³, Q value: 3.0, MI=20 g/10 min, melting point: 120° C., hexene copolymerization, flexural modulus: 600 MPa, hardness (HDD): 60), linear low density polyethylene, was prepared as a sheath component, and T200E (Toray Industries, Inc.; melting point: 250° C., limiting viscosity value (IV value): 0.64), polyethylene terephthalate, was prepared as a core component.

Using an eccentric core/sheath composite nozzle (600 holes), these two components were melt extracted at a sheath component/core component composite ratio (volume ratio) of 55/45 under the following conditions: spinning temperature of the sheath component: 260° C., spinning temperature of the core component: 300° C., nozzle temperature: 290° C. Thereby, a spun filament having an eccentricity ratio of 25% and a fineness of 6.8 dtex was obtained. When melt extruding, the discharge rate was 250 g/min and the pulling speed was 615 m/min.

The resulting spun filament was stretched to 2.6 times in hot water having a temperature of 80° C., thus forming a stretched filament having a fineness of about 3.3 dtex. Thereafter, in order to impart hydrophilicity to the stretched filament, 0.4 mass % of a hydrophilic fiber treating agent was added. Then, the stretched filament was subjected to machine crimping using a stuffing box crimper and provided with 12 crimps/25 mm. Then, the resulting filament was subjected to simultaneous annealing and drying, in a relaxed state, for 15 minutes using a hot air blowing device set to a temperature of 100° C. Thereafter, the filament was cut at a fiber length of 38 mm. Thus, a core/sheath composite fiber A-1 having three-dimensional crimp was obtained. The number of crimps measured in accordance with JIS L 1015 (2010) was 15.9 crimps/25 mm, and the crimping rate was 11.3%.

### Manufacture of Core/Sheath Composite Fiber A-2

Umerit® ZM076 (Ube-Maruzen Polyethylene Co., Ltd.; density: 0.931 g/cm³, MI=20 g/10 min, melting point: 120° C., hexene copolymerization, flexural modulus: 600 MPa, hardness (HDD): 60), linear low density polyethylene, and NOVATEC® LJ 802 (Japan Polyethylene Corporation; density: 0.918 g/cm³, MI=22 g/10 min, melting point: 106° C., flexural modulus: 130 MPa, hardness (HDD): 46), low density polyethylene, were prepared as a sheath component, and T200E (Toray Industries, Inc.; melting point: 250° C., limiting viscosity value (IV value): 0.64), polyethylene terephthalate, was prepared as a core component. In the sheath component, linear low density polyethylene (Umerit® ZM076) and low density polyethylene (NOVATEC® LJ 802) were mixed at the mass ratio of 85/15 (LLDPE/LDPE). Core/sheath composite fiber A-2 was prepared according to the same procedure and conditions as those employed in the manufacture of the core/sheath composite fiber A-1. The number of crimps measured in accordance with JIS L 1015 (2010) was 12.9 crimps/25 mm, and the crimping rate was 10.4%.

### Manufacture of Core/Sheath Composite Fiber B-1

NOVATEC® HE 490 (Japan Polyethylene Corporation; density: 0.956 g/cm³, MI=22 g/10 min, melting point: 133° C.), high density polyethylene, was prepared as a sheath component, and T200E, polyethylene terephthalate, was prepared as a core component. Core/sheath composite fiber B-1 was prepared according to the same procedure and conditions, with the exception of cutting the obtained filament at a fiber length of 51 mm, as those employed in the manufacture of the core/sheath composite fiber A-1. The number of crimps measured in accordance with JIS L 1015 (2010) was 16.2 crimps/25 mm, and the crimping rate was 12.1%.

### Manufacture of Core/Sheath Composite Fiber B-2

Core/sheath composite fiber B-2 was prepared according to the same procedure and conditions as those employed in the manufacture of the core/sheath composite fiber B-1, with the exception that the number of crimps measured in accordance with JIS L 1015 (2010) was 16.9 crimps/25 mm, and the crimping rate was 14.2%. A treatment agent for treating the core/sheath composite fiber B-2 was more hydrophilic and more resistant to removal than that for treating the core/sheath composite fiber A-2.

### Examples 1 to 3 and Comparative Examples 1 to 5

Using the core/sheath composite fiber A-1, first fibrous webs having the basis weights shown in Table 1 were fabricated using a parallel carding machine. Using the core/sheath composite fiber B-1, second fibrous webs having the basis weights shown in Table 1 were fabricated for Examples 1 to 3 and Comparative Examples 1 to 5 using a parallel carding machine. Complex webs were fabricated by laminating the first fibrous webs and the second fibrous webs and each of the complex webs were subjected to thermal treatment at the temperatures shown in Table 1. The thermal treatment was performed using a hot air through-type thermal treatment apparatus in the temperature shown in Table 1. The complex web was placed on the conveyor belt so that the surface of the first fiber layer is in contact with the breathable conveyor belt of the thermal treatment apparatus. Thermal bonded nonwovens were obtained via the thermal treatment.

The resulting nonwovens were evaluated as described below.

The thickness was measured using a caliper gauge (CR-60A, manufactured by Daiei Kagaku Seiki Mfg. Co., Ltd.) under conditions where a 2.94 cN load was applied to a 1 cm² test sample, and indicated in Table 1.

In order to evaluate surface tactile sensation, and softness and bulkiness recovery characteristics (resiliency) in the thickness direction, surface property and compression property measurements and evaluations were performed based on the KES (Kawabata Evaluation System) described under TEST METHODS above.

Specifically, standard mean deviation of surface roughness mean (“SMD”) was measured using a KES-SE friction sensitivity tester (manufactured by Kato Tech Co., Ltd.), and indicated in Table 1. When measuring, the surface of the first fiber layer was used as the measuring surface, a static load of 25 gf was placed on the friction block, and the friction block was moved in a direction parallel to the machine direction of the nonwoven at a movement speed of 1 mm/sec.

Work of compression (“WC”) and resiliency of compression (“RC”) were measured from the load-displacement curve as the compression property values, and indicated in Table 1. The compression test and the measurement of the compression property values were conducted using a KES-G5 compression tester (manufactured by Kato Tech Co., Ltd.). When measuring, a circular pressure plate with an area of 2 cm² was used as the compression block and the SENS was set to 2 and the DEF sensitivity to 20. The compression block was pressed against the nonwoven and compressed at a compression speed of 0.02 cm/sec until the load was 50 gf/cm². After the load reached 50 gf/cm², compression was removed so that the movement speed of the compression block was 0.02 cm/sec, and the compression property values described above were measured.

TABLE 1

| | | | | | Com | Com | Com | Com | Com |
|---|---|---|---|---|---|---|---|---|---|
| | | Ex 1 | Ex 2 | Ex 3 | Ex 1 | Ex 2 | Ex 3 | Ex 4 | Ex 5 |
| 1st layer | Fiber | A-1 | A-1 | A-1 | A-1 | A-1 | A-1 | B-1 | B-1 |
| | Basis weight (g/m²) | 25 | 25 | 25 | 25 | 25 | 25 | 25 | 25 |
| 2nd layer | Fiber | B-1 | B-1 | B-1 | A-1 | A-1 | A-1 | B-1 | B-1 |
| | Basis weight (g/m²) | 25 | 25 | 25 | 25 | 25 | 25 | 25 | 25 |
| heat treatment | Temperature (° C.) | 130 | 135 | 140 | 130 | 135 | 140 | 130 | 135 |
| Nonwoven | 1st layer L/H | 4.72 | 5.20 | 4.41 | 4.81 | 5.23 | 4.50 | 2.64 | 3.00 |
| Properties | 2nd layer L/H | 3.46 | 3.84 | 3.78 | 4.89 | 4.12 | 4.10 | 3.62 | 3.71 |
| | 1st(L/H)/2nd(L/H) | 1.36 | 1.35 | 1.17 | 0.98 | 1.27 | 1.10 | 0.73 | 0.81 |
| | Basis weight (g/m²) | 51.7 | 48.1 | 49.8 | 50.5 | 50 | 49.4 | 50.3 | 50.3 |
| | Thickness (mm) | 4.94 | 4.63 | 4.48 | 3.41 | 3.45 | 3.37 | 5.32 | 5.05 |
| | Specific volume (cm³/g) | 95.6 | 96.3 | 90.0 | 67.5 | 69.0 | 68.2 | 105.8 | 100.4 |
| | SMD (μm) | 1.38 | 1.38 | 1.23 | 1.62 | 1.30 | 1.45 | 2.00 | 1.92 |
| | WC (gf · cm/cm²) | 3.877 | 3.880 | 3.685 | 2.595 | 2.731 | 2.402 | 4.033 | 3.884 |
| | RC (%) | 62.2 | 62.2 | 63.3 | 65.6 | 64.2 | 66.9 | 59.4 | 59.5 |

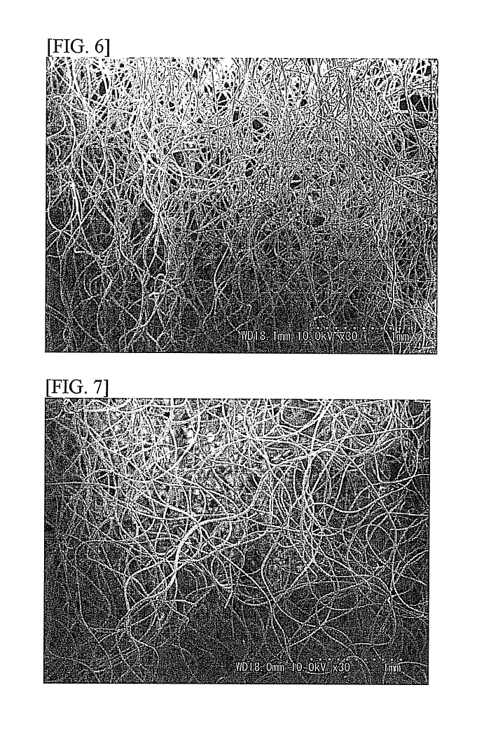

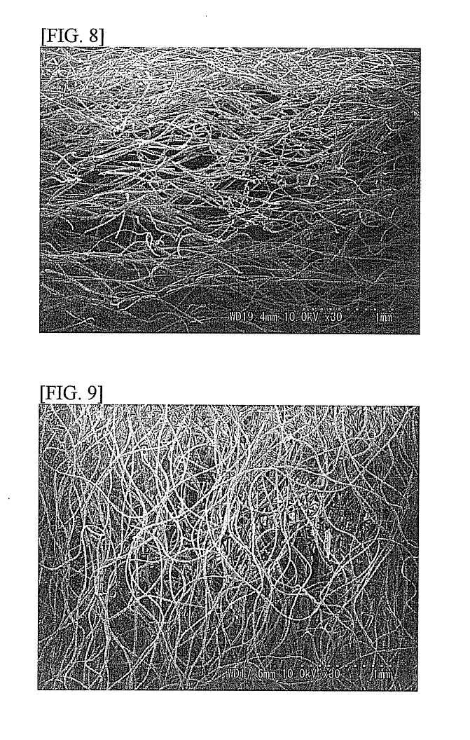

Nonwovens of Example 1, Comparative examples 1 and 4 were closely observed via electronic microscope image (magnification: 30×, Hitachi, S3500N-2). In FIG. 5, compression in the thickness direction varies. The first fiber layer is more compact and the second fiber layer is less compact. The difference in fiber density of the layers is also clear from the surface conditions of each of the layers. In FIG. 6 showing the surface of fiber A-1 layer, the three-dimensional crimp of the first core/sheath composite fiber (core/sheath composite fiber) is weak and has a compact structure in which not only has flattening advanced, but space between the fibers is narrow. In contrast, space between the fibers shown in FIG. 7 showing the surface of fiber B-1 layer is wide, and the structure thereof is less than dense fiber A-1 layer.

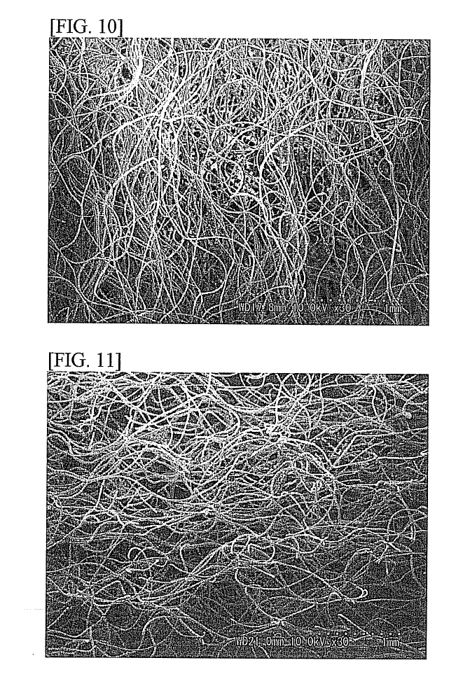

In FIG. 8, image of a cross-section of a nonwoven of Comparative Example 1 manufactured from only fiber A-1, it was observed that the nonwoven of Comparative Example 1 was formed with a compact structure in the entire nonwoven. From FIG. 9 and FIG. 10, it was observed that there is no difference between the crimped shapes of the fibers of both surfaces of Comparative Example 1. In FIG. 11, image of a cross-section of a nonwoven of Comparative Example 4 manufactured from only fiber B-1, it was observed that the nonwoven of Comparative Example 4 was not formed with a compact structure.

### Examples 4 and 5, and Comparative Examples 6 and 7

As for Examples 4 and 5, nonwovens were prepared using Fiber A-1 and Fiber B-1 according to the manufacturing method described in Examples 1 to 3 and Comparative Examples 1 to 5 except using a random carding machine instead of a parallel carding machine and heat treatment temperature of 133° C. As for Comparative Examples 6 and 7, nonwovens were prepared using Fiber A-1 only according to the same manufacturing method of Examples 4 and 5. Each nonwoven was winded a roll. Example 4 and Comparative Example 6 were nonwoven samples from top of nonwoven roll, and Example 5 and Comparative Example 7 were nonwoven samples from bottom of nonwoven roll. Measurement was conducted 2 days later sample was released from the roll.

Estimated compressible thickness, work of compression, and resilience of compression of nonwoven according to the present invention were measured using MTS Criterion Model 42 (MTS Systems Corporation) having a 10N or 100N load cell and a circular compression platen of 16 mm diameter, as indicated in Table 2.

Specifically, estimated compressible thickness of nonwoven was measured using MTS Criterion Model 42 (MTS Systems Corporation) as the compression platen press the nonwoven sample until the load reaches 2N force. Estimated compressible thickness means the distance that the MTS crosshead travels from force 0.01N (contact) to force 2N (maximum force applied to the sample).

When measuring work of compression, the compression platen was pressed against the nonwoven (sample size: 2.54 cm×2.54 cm) at a crosshead speed 0.02 mm/s (1.2 mm/min); Data Acquisition Rate: 100 Hz; maxim compressive force applied to the samples: 2N.

TABLE 2

| | | Ex 4 | Ex 5 | Com Ex 6 | Com Ex 7 |
|---|---|---|---|---|---|
| 1st | Fiber | A-1 | A-1 | A-1 | A-1 |
| layer | Basis weight (g/m²) | 20 | 20 | 20 | 20 |
| 2nd | Fiber | B-1 | B-1 | A-1 | A-1 |
| layer | Basis weight (g/m²) | 30 | 30 | 30 | 30 |
| Properties | Basis weight (g/m²) | 50 | 50 | 50 | 50 |
| | Estimated | 2.51 | 2.69 | 1.37 | 1.32 |
| | Compressible | | | | |
| | Thickness (mm) | | | | |
| | WC (gf · cm/cm²) | 2.52 | 2.59 | 1.22 | 1.06 |
| | RC (%) | 62.3 | 65.3 | 72.4 | 69.6 |

### Examples 6 to 13

As for Examples 6 to 13, nonwovens were prepared using Fiber A-2 and Fiber B-2. Standard mean deviation of surface roughness (SMD) of the first fiber layer, estimated compressible thickness, work of compression, and resilience of compression of nonwoven were measured as described in Examples 1 to 3 and indicated in Table 3. In Example 13, SMD of the second fiber layer was measured in addition to SMD of the first fiber layer.

TABLE 3

| | | Ex 6 | Ex 7 | Ex 8 | Ex 9 | Ex 10 | Ex 11 | Ex 12 | Ex 13 |
|---|---|---|---|---|---|---|---|---|---|
| 1st layer | Fiber | A-2 | A-2 | A-2 | A-2 | A-2 | A-2 | A-2 | B-2 |
| | Basis weight (g/m²) | 15 | 20 | 30 | 35 | 30 | 15 | 20 | 25 |
| 2nd layer | Fiber | B-2 | B-2 | B-2 | B-2 | B-2 | B-2 | B-2 | A-2 |
| | Basis weight (g/m²) | 15 | 20 | 30 | 35 | 20 | 35 | 30 | 25 |
| heat treatment | Temperature (° C.) | 135 | 135 | 135 | 135 | 135 | 135 | 135 | 135 |
| Nonwoven | 1st layer L/H | 3.63 | 4.65 | 3.42 | 3.70 | 4.09 | 4.10 | 3.29 | 1.76 |
| Properties | 2nd layer L/H | 1.97 | 2.28 | 2.55 | 2.27 | 3.05 | 2.39 | 2.64 | 2.39 |
| | 1st(L/H)/ 2nd(L/H) | 1.84 | 2.04 | 1.34 | 1.63 | 1.34 | 1.71 | 1.24 | 0.74 |
| | Basis weight (g/m²) | 30.3 | 40.3 | 60.3 | 69.4 | 48.7 | 49.2 | 50.5 | 49.7 |
| | Thickness (mm) | 3.37 | 4.46 | 6.15 | 6.69 | 4.84 | 5.31 | 5.36 | 4.64 |
| | Specific volume (cm³/g) | 111.2 | 110.6 | 102.0 | 96.4 | 99.5 | 108.0 | 106.1 | 93.4 |
| | SMD (μm) | 2.86 | 2.39 | 1.22 | 1.29 | 1.29 | 1.73 | 1.38 | 1st layer: 2.40 |
| | | | | | | | | | 2nd layer: 2.32 |
| | WC (gf · cm/cm²) | 2.868 | 3.467 | 4.212 | 4.853 | 3.675 | 4.011 | 4.251 | 3.613 |
| | RC (%) | 57.1 | 60.0 | 62.9 | 67.3 | 62.6 | 60.5 | 62.5 | 62.5 |

### Examples 14 and 15

As for Examples 14 and 15, nonwovens were prepared according to the manufacturing method described in Examples 4 and 5 except for using Fiber A-2 and Fiber B-2 and a random carding machine instead of a parallel carding machine and heat treatment temperature of 133° C. Each nonwoven was winded to a roll. Example 14 was nonwoven samples from top of nonwoven roll, and Example 15 was nonwoven samples from bottom of nonwoven roll. Measurement was conducted about 2 days later sample was released from the roll.

The resulting nonwovens were evaluated at the basis weight, and the L/H. The measurement procedure of each evaluation is the same as the measurement of example 1 to 5 that is as described above, and indicated in Table 4.

TABLE 4

| | | Ex 14 | Ex 15 |
|---|---|---|---|
| 1st layer | Fiber | A-2 | A-2 |
| | Basis weight | 20 | 20 |
| | (g/m²) | | |
| 2nd layer | Fiber | B-2 | B-2 |
| | Basis weight | 30 | 30 |
| | (g/m²) | | |
| heat | Temperature | 133 | 133 |
| treatment | (° C.) | | |
| Nonwoven | 1st fiber | 4.61 | 4.65 |
| Properties | layer L/H | | |
| | 2nd fiber | 2.47 | 2.28 |
| | layer L/H | | |
| | 1st(L/H)/ | 1.87 | 2.04 |
| | 2nd(L/H) | | |
| | Basis weight | 50.4 | 40.3 |
| | (g/m²) | | |

### Example 16

A sanitary napkin having a nonwoven topsheet, an air-laid tissue secondary layer, absorbent core, and a backsheet were prepared. A nonwoven for the topsheet was manufactured using the fiber combination (20 g/m² of fiber A-1 and 30 g/m² of fiber B-1) and process substantially identical to the process for Example 4 nonwoven.

A topsheet was separated from a sanitary napkin after a sample of 80 mm×60 mm was cut from middle of the napkin. The sample was sprayed with freeze-it spray and topsheet was peeled off from secondary layer. A topsheet sample was let to rest about 2 hours before its thickness was measured.

Thickness of a sample was measured using a caliper gauge model No. HDS-8″M manufactured by Mitutoyo Corporation Japan with a probe having a foot of 25 mm diameter. The caliper gauge is capable of measuring thickness with a 0.01 mm tolerance. A topsheet was positioned on a flat surface. Probe was manually lowered to touch the surface of the topsheet by turning knob on the equipment. The shaft and foot was setup to deliver approximately 1.5 g of force for a pressure of 0.004 psi to the sample. Distance moved by the gauge was recorded directly from the gauge. 10 topsheet samples were measured and obtained average thickness of 1.94 mm (standard deviation: 0.1388) at pressure 0.004 psi. From the average thickness and nonwoven basis weight of 50 g/m², specific volume of the nonwoven topsheet was 39 cm³/g.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A sheet for an absorbent article comprising a nonwoven comprising
a first fiber layer comprising a first core/sheath composite fiber having three-dimensional crimp, wherein a sheath component of the first core/sheath composite fiber comprises linear low density polyethylene, a core component of the first core/sheath composite fiber comprises thermoplastic resin having a melting point of at least about 20° C. higher than a melting point of the linear low density polyethylene, and the center of gravity of the core component is offset from the center of gravity of the fiber;
a second fiber layer comprising a second core/sheath composite fiber having three-dimensional crimp, wherein a sheath component of the second core/sheath composite fiber comprises high density polyethylene, a core component of the second core/sheath composite fiber comprises thermoplastic resin having a melting point of at least about 20° C. higher than a melting point of the high density polyethylene, and the center of gravity of the core component is offset from the center of gravity of the fiber;
wherein at least a portion of the first and the second core/sheath composite fibers is thermally bonded via the sheath components of the first and the second core/sheath composite fibers.

2. The sheet for an absorbent article according to claim 1, wherein said sheath component of the first core/sheath composite fiber in the first fiber layer further comprises low density polyethylene.

3. The sheet for an absorbent article according to claim 1, wherein said first fiber layer is denser than the second fiber layer.

4. The sheet for an absorbent article according to claim 1, wherein said sheath component of the first core/sheath composite fiber comprises at least 60 mass % of linear low density polyethylene by the mass of said sheath component.

5. The sheet for an absorbent article according to claim 1, wherein said sheath component of the second core/sheath composite fiber comprises at least 60 mass % of high density polyethylene by the mass of said sheath component.

6. The sheet for an absorbent article according to claim 1, wherein the ratio of basic weight of the first fiber layer to the basic weight of the second fiber layer is in the range of about 70/30 to about 20/80.

7. The sheet for an absorbent article according to claim 1, wherein fiber lengths of the first and the second core/sheath composite fibers are not more than about 100 mm.

8. The sheet for an absorbent article according to claim 1, wherein said first fiber layer is less hydrophilic than the second fiber layer.

9. The sheet for an absorbent article according to claim 1, wherein a crimping degree of the three-dimensional crimp of the second core/sheath composite fiber is greater than a crimping degree of the three-dimensional crimp of the first core/sheath composite fiber.

10. The sheet for an absorbent article according to claim 1, wherein a surface of the first fiber layer which does not contact with the second fiber layer has a standard mean deviation of surface roughness (SMD) of about 4 μm or less.

11. The sheet for an absorbent article according to claim 1, wherein the L/H of the first core/sheath composite fiber in the first fiber layer to the L/H of the second core/sheath composite fiber in the second fiber layer is at least 1.05 when L is a distance between bottoms of two adjacent valleys of a three-dimensional crimp and H is a height from an apex of a peak of the three-dimensional peak to a line between the bottoms of the two adjacent valleys of the three-dimensional crimp.

12. A method for manufacturing a nonwoven comprising:
forming a first fibrous web comprising a first core/sheath composite fiber having three-dimensional crimp, wherein a sheath component of the first core/sheath composite fiber comprises linear low density polyethylene, a core component of the first core/sheath composite fiber comprises thermoplastic resin having a melting point of at least about 20° C. higher than a melting point of the linear low density polyethylene, and the center of gravity of the core component is offset from the center of gravity of the fiber;
forming a second fibrous web comprising a second core/sheath composite fiber having three-dimensional crimp, wherein a sheath component of the second core/sheath composite fiber comprises high density polyethylene, a core component of the second core/sheath composite fiber comprises thermoplastic resin having a melting point of at least about 20° C. higher than a melting point of the high density polyethylene, and the center of gravity of the core component is offset from the center of gravity of the fiber;
forming a complex fibrous web by laminating the first fibrous web and the second fibrous web; and
subjecting the complex fibrous web to thermal treatment in order to thermally bond at least a portion of the fibers via the sheath components of the first core/sheath composite fiber and the second core/sheath composite fiber.

13. The method for manufacturing a nonwoven according to claim 12, wherein the thermal treatment is performed using a hot air through-type thermal treatment apparatus comprising a conveying support.

14. The method for manufacturing a nonwoven according to claim 13, wherein the first fibrous web is placed on the conveying support so that the first fiber layer contacts the conveying support during the thermal treatment.

15. An absorbent article comprising
a topsheet; and
a liquid impervious backsheet joined to the topsheet,
wherein the topsheet comprises nonwoven comprising
a first fiber layer comprising a first core/sheath composite fiber having three-dimensional crimp, wherein a sheath component of the first core/sheath composite fiber comprises linear low density polyethylene, a core component of the first core/sheath composite fiber comprises thermoplastic resin having a melting point of at least about 20° C. higher than a melting point of the linear low density polyethylene, and the center of gravity of the core component is offset from the center of gravity of the fiber;
a second fiber layer comprising a second core/sheath composite fiber having three-dimensional crimp, wherein a sheath component of the second core/sheath composite fiber comprises high density polyethylene, a core component of the second core/sheath composite fiber comprises thermoplastic resin having a melting point of at least about 20° C. higher than a melting point of the high density polyethylene, and the center of gravity of the core component is offset from the center of gravity of the fiber;
wherein at least a portion of the first and the second core/sheath composite fibers is thermally bonded via the sheath components of the first and the second core/sheath composite fibers.

16. The absorbent article according to claim 15, wherein said topsheet has a specific volume in the range of about 10 cm³/g to about 60 cm³/g.

17. The absorbent article according to claim 15, wherein, the first fiber layer is positioned on a side in contact with the skin of a wearer.

18. The absorbent article according to claim 15 further comprising an absorbent core placed between the topsheet and the backsheet.

19. The absorbent article according to claim 15, wherein a surface of the first fiber layer which does not contact with the second fiber layer has a standard mean deviation of surface roughness (SMD) of about 4 μm or less.

20. A nonwoven comprising
a first fiber layer comprising a first core/sheath composite fiber having three-dimensional crimp, wherein a sheath component of the first core/sheath composite fiber comprises linear low density polyethylene, a core component of the first core/sheath composite fiber comprises thermoplastic resin having a melting point of at least about 20° C. higher than a melting point of the linear low density polyethylene, and the center of gravity of the core component is offset from the center of gravity of the fiber;
a second fiber layer comprising a second core/sheath composite fiber having three-dimensional crimp, wherein a sheath component of the second core/sheath composite fiber comprises high density polyethylene, a core component comprises thermoplastic resin having a melting point of at least about 20° C. higher than a melting point of the high density polyethylene, and the center of gravity of the core component of the second core/sheath composite fiber is offset from the center of gravity of the fiber;
wherein at least a portion of the first and the second core/sheath composite fibers is thermally bonded via the sheath components of the first and the second core/sheath composite fibers.
